(19) United States
(12) Patent Application Publication
(10) Pub. No.: US 2012/0135368 A1
(43) Pub. Date: May 31, 2012

(54) Modified-ouput fiber optic tips

(76) Inventors: Ioana M. Rizoiu, San Clemente, CA (US); Dmitri Boutoussov, Dana Point, CA (US); Jeffrey W. Jones, Robertson, WY (US); Peter Chueh, Irvine, CA (US)

(21) Appl. No.: 12/020,455

(22) Filed: Jan. 25, 2008

Related U.S. Application Data
(60) Provisional application No. 60/898,022, filed on Jan. 26, 2007.
(60) Provisional application No. 60/920,711, filed on Mar. 28, 2007.

(51) Int. Cl. A61N 5/067; A61B 17/00; A61B 18/22

(52) U.S. Cl. 433/29; 606/16

(57) ABSTRACT

A laser handpiece is disclosed, including a shaped fiber optic tip having a side-firing output end with a double bevel-cut shape. The shaped fiber optic tip can be configured to side-fire laser energy in a direction away from a laser handpiece and toward sidewalls of a treatment or target site.

### CROSS-REFERENCE TO RELATED APPLICATIONS

The application claims priority to U.S. Provisional application Ser. No. 11/898,022, filed on Jan. 26, 2007 (Att. Docket BI9827CIPPR) and entitled MODIFIED-OUTPUT FIBER OPTIC TIPS, and to U.S. Provisional Application No. 60/920,711, filed on Mar. 28, 2007 (Att. Docket BI9827CIPPR3) and entitled MODIFIED-OUTPUT FIBER OPTIC TIPS, the entire contents of both which are incorporated herein by reference. The application is related to U.S. application Ser. No. 11/033,441, filed on Jan. 10, 2005, the contents of which are incorporated herein by reference.

### BACKGROUND OF THE INVENTION

1. Field of the Invention

The present invention relates generally to medical devices and, more particularly, to fiber optic tips for delivering electromagnetic radiation.

2. Description of the Related Art

Fiber optics have existed in the prior art for delivering electromagnetic radiation. Radiation delivery systems are typically used to transport electromagnetic radiation from electromagnetic energy sources to treatment sites. One common radiation delivery system can comprise a cylindrically-shaped fiber optic tip from which electromagnetic radiation is emitted in a direction toward the treatment site.

In certain applications, radiation delivery systems can be engineered to generate predetermined beam shapes and spatial energy distributions. The energy distribution of a simple delivery system, comprising a fiber optic tip, can be described as having a circular illumination area, with a so-called Gaussian distribution of beam intensities being spatially distributed within the output beam pattern or illuminated area. For instance, the output beam pattern from a fiber optic tip can comprise a central high-intensity area or “hot spot” surrounded by peripheral areas of lower intensity.

Regarding energy distributions, some beam profiling applications can require or would be optimized with radiation delivery systems capable of generating illumination distributions that vary across parts or all of the illumination area surrounding the output of the radiation delivery system. Moreover, it may also be desirable to generate non-circular illumination areas, or to generate electromagnetic radiation having predetermined energy distributions across a non-planar illumination area. Use of laser radiation having a relatively uniform power distribution over a particularly shaped area can be a practical task for multiple medical applications.

### SUMMARY OF THE INVENTION

The present invention provides optical arrangements and relatively compact medical laser instruments to deliver electromagnetic radiation to treatment sites with power distributions that may vary in a non-Gaussian distribution fashion, compared to cylindrical output fibers, across parts or all of the illumination area surrounding the output waveguide. The illumination areas may comprise non-circular or curved surfaces, such as cavities, in which case substantial output power densities can be concentrated on sidewalls of the illumination areas. The electromagnetic radiation can comprise laser radiation, and the treatment site can comprise tissue to be treated.

The various embodiments of the present invention may include or address one or more of the following objectives. One objective is to provide a fiber optic tip having a shaped fiber optic output end (i.e., a fiber optic output end not consisting only of a planar surface orthogonal to the fiber optic axis) for delivery of electromagnetic radiation, wherein electromagnetic radiation exiting the fiber optic output end is not concentrated along the fiber optic axis. Another objective is to provide a fiber optic output end having an emission characteristic whereby electromagnetic radiation exiting the fiber optic output end is relatively weak along the fiber optic axis. Yet another object is to provide a fiber optic output end wherein all waveguide modes experience a majority or total internal reflection on a first surface of the fiber optic output end and go out through an opposite surface of the fiber optic output end. Still another objective is to provide a apparatus for directing laser energy and optionally fluid to different target sites through different reflections within a fiber conduit and from the fiber conduit to the output end or sites, wherein different energy distributions can be provided to different treatment surfaces surrounding or in a vicinity to the fiber conduit at the same time.

While the apparatus and method have or will be described for the sake of grammatical fluidity with functional explanations, it is to be expressly understood that terms in the additional disclosure in claims format, unless expressly formulated under 35 USC 112, are not to be construed as necessarily limited in any way by the construction of “means” or “steps” limitations, but are to be accorded the full scope of the meaning and equivalents of the definition provided by such claims under the judicial doctrine of equivalents, and in the case where terms in the additional disclosure in claims format are expressly formulated under 35 USC 112 are to be accorded full statutory equivalents under 35 USC 112.

Any feature or combination of features described herein are included within the scope of the present invention provided that the features included in any such combination are not mutually inconsistent as will be apparent from the context, this specification, and the knowledge of one of ordinary skill in the art. For purposes of summarizing the present invention, certain aspects, advantages and novel features of the present invention have been described herein. Of course, it is to be understood that not necessarily all such aspects, advantages or features will be embodied in any particular embodiment of the present invention. Additional advantages and aspects of the present invention are apparent in the following detailed description and additional disclosure in claims format.

### BRIEF DESCRIPTION OF THE FIGURES

FIG. 1 is a cross-sectional view of a rotating handpiece;

FIG. 2 is a cross-sectional view of an alternative embodiment of the rotating handpiece;

FIG. 3 is a side elevation view of the rotating hand piece in a partially disassembled state;

FIGS. 3a-6 are other views of the structure;

FIG. 7 is a perspective view of the loading tool, fiber tip fluid output device, and handpiece head in a disassembled configuration;

FIG. 8 is an end view of the loading tool, taken along the line 8-8 of FIG. 7;

FIG. 9 is a perspective view of the fiber tip fluid output device partially secured onto the loading tool, just before insertion of the fiber tip fluid output device into the handpiece head;

FIG. 10 is a cross-sectional view of shaped fiber optic tip having a conical side-firing output end in accordance with an embodiment of the present invention;

FIG. 11 shows calculation considerations pertaining to determination of a cone angle of a fiber optic end of a radiation emitting apparatus;

FIG. 12 shows a side firing tip comprising a shaped fiber optic tip having dual, opposing bevel-cut side-firing output ends according to an embodiment of the present invention;

FIG. 12A shows a side firing tip comprising a shaped fiber optic tip having three bevel-cuts according to an embodiment of the present invention;

FIGS. 12B and 13 show other implementations of a side firing tip comprising a shaped fiber optic tip with dual, opposing bevel-cut side-firing output ends according to an embodiment of the present invention;

FIG. 13B shows a side firing tip comprising a shaped fiber optic tip having dual bevel-cut side-firing output ends with an arched shape according to another embodiment of the present invention;

FIG. 14 shows another implementation of a side firing tip comprising a shaped fiber optic tip having three bevel-cuts and a truncated distal surface according to an embodiment of the present invention;

FIG. 15 shows an implementation of a side firing tip comprising a shaped fiber optic tip as in FIG. 12B and having a truncated distal surface, according to an embodiment of the present invention;

FIGS. 16-18 show side firing tips comprising shaped fiber optic tips having three bevel-cuts according to embodiments of the present invention;

FIG. 19 shows a side firing tip comprising a shaped fiber optic tip having dual bevel-cut side-firing output ends similar to the configuration of FIG. 18, wherein the length of the side firing tip is less than a length of an initial structure as in FIG. 18, according to an embodiment of the present invention;

FIGS. 20-27 show side firing tips comprising shaped fiber optic tips having bevel-cut side-firing output ends similar to the configuration of FIG. 18, wherein one or more dimensions of the side firing tips vary from that or those of an initial structure as in FIG. 18, or are less symmetrical, according to embodiments of the present invention;

FIG. 28 shows an implementation of a side firing tip comprising an off-axis shaped fiber optic tip, with a truncated distal surface, according to an embodiment of the present invention;

FIG. 29 shows an implementation of a side firing tip comprising a shaped fiber optic tip with three beveled surfaces, and a fourth beveled and nearly-truncated distal surface, according to an embodiment of the present invention;

FIG. 30 shows an implementation of a side firing tip comprising a shaped fiber optic tip with three beveled surfaces and a fourth beveled distal surface, according to an embodiment of the present invention;

FIG. 30 shows side firing tips comprising shaped fiber optic tips having three bevel-cuts according to embodiments of the present invention; and

FIGS. 31, 32A, 32B, 32C, 33A, 33B, 33C and 33D show implementations of side firing tips comprising shaped fiber optic tips with truncated and non-truncated distal surfaces according to embodiments of the present invention.

### DETAILED DESCRIPTION OF THE PRESENT INVENTION

Reference will now be made in detail to certain embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the additional disclosure in claims format.

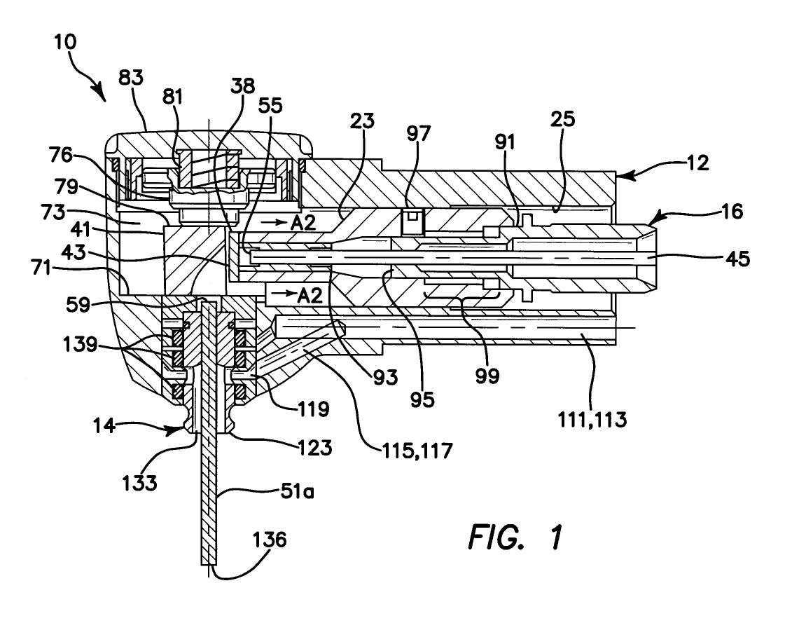

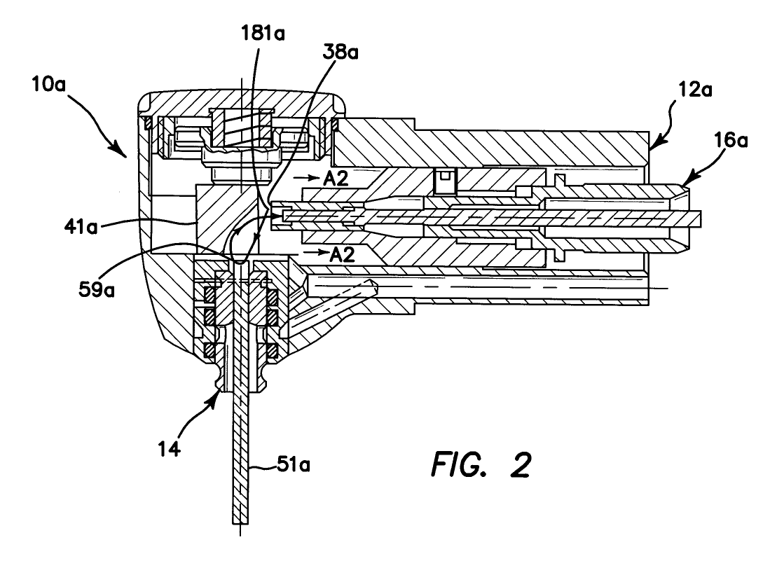

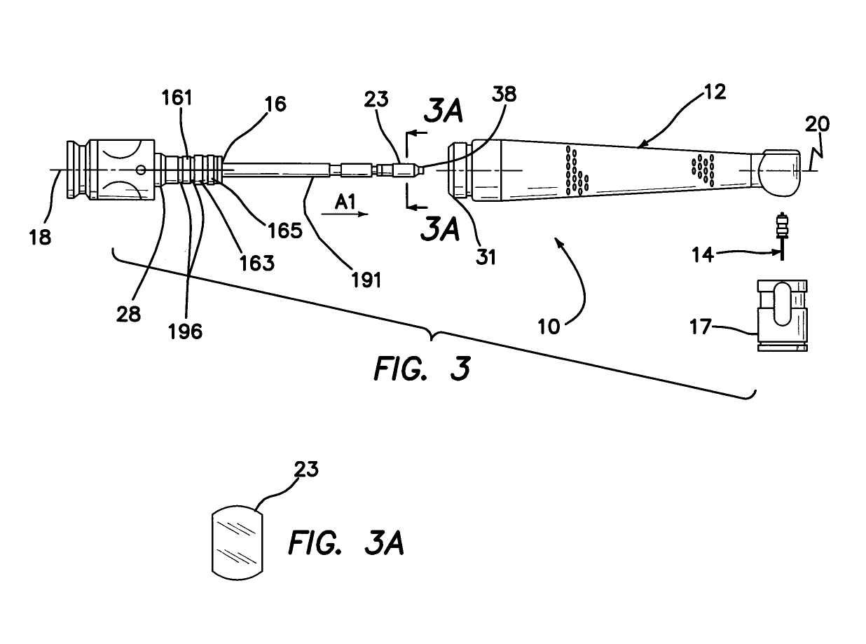

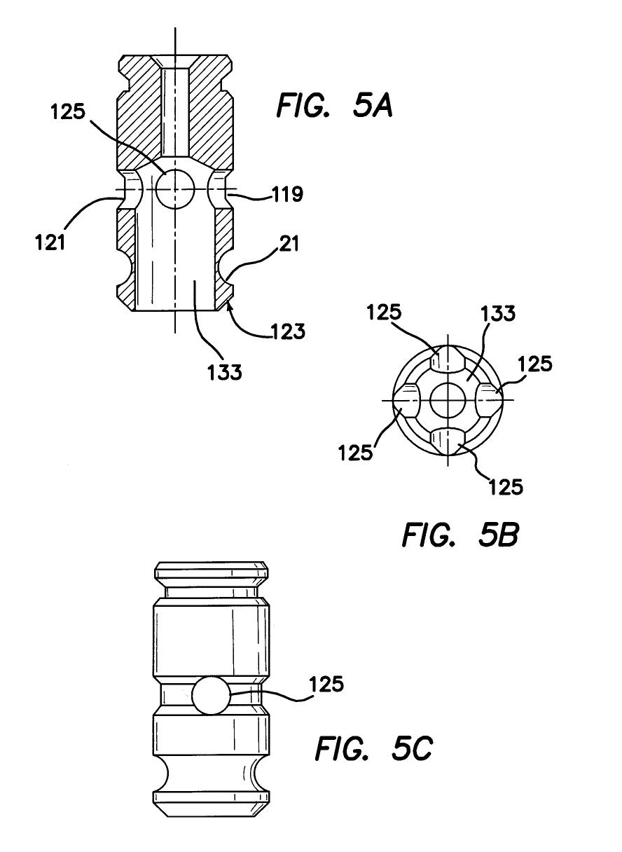

Referring more particularly to the drawings, FIG. 1 illustrates a cross sectional view of the rotating handpiece 10. The rotating handpiece comprises a handpiece head 12, a fiber tip fluid output device 14, and a removable trunk fiber assembly 16. These components can be seen in a partially disassembled state in FIG. 3, wherein the axis 18 of the removable trunk fiber assembly 16 is aligned with the axis 20 of the handpiece head 12 for insertion into the handpiece head 12. Once the axis 18 of the removable fiber assembly 16 is aligned with the axis 20 of the handpiece 12, the removable trunk fiber assembly 16 is moved in the direction of the arrow A1 into the handpiece head 12, while the axes 18 and 20 are maintained in approximate alignment. The contacting surface of the outer surface of the chuck 23 engages the inner surface 25 of the rotating handpiece 10, to thereby ensure alignment of the axis 18 of the removable trunk fiber assembly 16 and the axis 20 of the handpiece head 12. As the removable trunk fiber assembly 16 is inserted further in the direction A1 into the handpiece 12, the abutting surface 28 engages with a corresponding abutting surface (not shown) within the collar 31 of the handpiece head 12. The corresponding abutting surface 28 can be constructed to snap with the abutting surface 31, as the removable trunk fiber assembly 16 is fully inserted into the handpiece head 12. Any type of locking engagement between the abutting surface 28 and a corresponding abutting surface within the collar 31, as known in the art, may be used to ensure that the removable trunk fiber assembly 16 is always inserted the same distance into the handpiece head 12. As shown in FIG. 1, the distal tip 38 of the removable trunk fiber assembly 16 is brought into close proximity with the parabolic mirror 41. In the illustrated embodiment, the distal tip 38 of the removable trunk fiber assembly 16 comprises a window 43 for protecting the trunk fiber optic 45 from contaminants, such as water. In the alternative embodiment shown in FIG. 2, the distal tip 38a is not protected with a window. As shown in FIG. 1, the fiber tip 51 of the fiber tip fluid output device 14 is also accurately placed in close proximity to the parabolic mirror 41. A loading tool 17 can be used to assist in the placement of the fiber tip fluid output device 14 into the handpiece head 12, as discussed below with reference to FIGS. 5 and 7-9. Electromagnetic radiation exiting from the output end 55 of the trunk fiber optic 45 is collected by the parabolic mirror 41 and, subsequently, reflected and focused onto the input end 59 of the fiber tip 51.

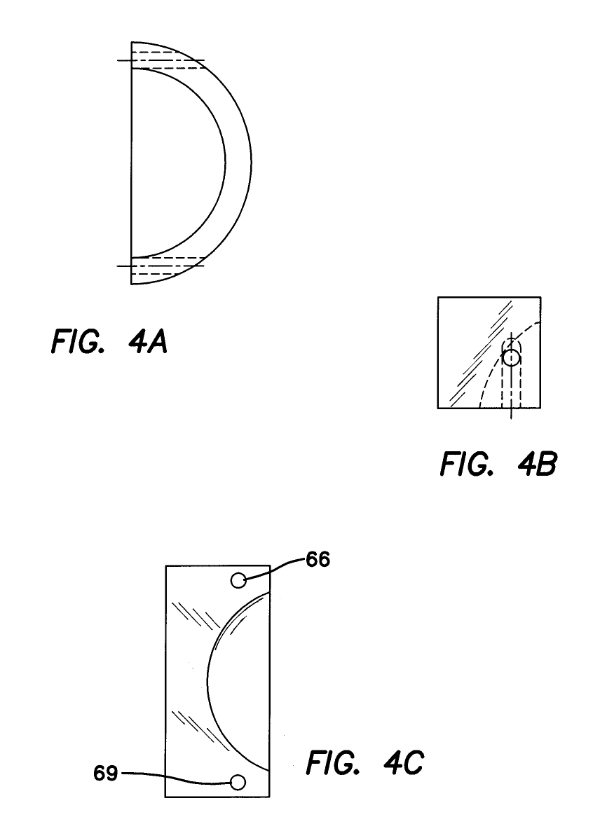

In one embodiment, the electromagnetic radiation exiting from the output end 55 of the trunk fiber optic 45 comprises a wavelength on the order of 3 microns. In other embodiments, electromagnetic radiation can be supplied at wavelengths from about 0.4 micron to about 11 microns, and in typical embodiments from about 0.4 micron to about 3 microns, from a light source such as a plasma arc lamp, a LED, or a laser having a continuous wave (CW) or pulsed mode of operation. The material of the parabolic mirror 41 is selected to provide an efficient reflection and focusing into the input end 59. As presently embodied, the electromagnetic radiation is generated from an Er:YSGG laser, and the material of the parabolic mirror 41 comprises a gold plating to provide reflectivity of approximately 99.9 percent. Other materials may be selected in accordance with design parameters. Other reflective surfaces and materials for the parabolic mirror 41 may be selected, in accordance with the laser being used and the desired efficiency of reflection. For example, if a lower reflectivity is selected, then additional cooling may be needed for the parabolic mirror 41 (such as a greater flow rate of cooled and/or filtered air across the surface of the parabolic mirror 41). FIGS. 4a, 4b and 4c illustrate various views of the parabolic mirrors 41 of the presently illustrated embodiment. The flat surface of the parabolic mirror 41, which is closest to the fiber tip 51, can be provided with two recessed areas 66 and 69. These two recessed areas mate with corresponding protrusions (not shown) on the floor 71 of the internal chamber 73 of the handpiece head 12. A spring loaded plunger 76 presses against the upper surface 79 of the parabolic mirror 41 under the pressure of the spring 81. A screw cap 83 holds the spring 81 against the spring loaded plunger 76. The combination of the spring loaded plunger 76, the recessed areas 66,69 of the parabolic mirror 41, and the corresponding protrusions on the floor 71, together, accurately align the parabolic mirror 41 for efficient coupling of electromagnetic radiation between the output end 55 of the trunk fiber optic 45 and the input end 59 of the fiber tip 51. In modified embodiments, either or both of the output end 55 of the trunk fiber optic 45 and the input end 59 of the fiber tip 51 is/are provided with an anti-reflective coating. Although it may be preferred in certain implementations to have the trunk fiber optic 45 perfectly aligned in relation to the parabolic mirror 41 and the fiber tip 51, the alignment between these three elements is seldomlv perfect. In the presently illustrated embodiment, the misalignment of the axis of the trunk fiber optic 45 and the axis of the fiber tip 51 is within plus or minus 1 percent error.

In a modified embodiment, a pentaprism (five-sided prism) is used instead of the parabolic mirror 41 for coupling the trunk fiber optic 45 to the fiber tip 51. In addition to slight misalignment of the axis of the trunk fiber optic 45, slight imperfections on the output end 55 of the trunk fiber optic 45 may also be present. The parabolic mirror 41 corrects for both of these slight errors, by collecting the electromagnetic radiation from the output end 55 of the front fiber optic 45 and, subsequently, focusing the electromagnetic radiation into the input end 55 of the fiber tip 51.

The parabolic mirror 41 may also comprise molypdium, in an exemplary embodiment. The clamp assembly 91 operates to firmly grip and hold the trunk fiber optic 45. In the presently illustrated embodiment, the clamp assembly 91 is provided with at least one slit, which extends from the distal end 93 of the clamp assembly 91 to a region 95 just distal of the set screw 97. As presently embodied, the at least one slit extending from the distal end 93 to the region 95 just distal of the set screw 97 comprises two slits, which are adapted to allow the clamp assembly 91 to be compressed by the chuck 23 onto the trunk fiber optic 45. The chuck 23 thus presses against the portion of the clamp assembly 91, wherein the portion is defined between the distal end 93 and the region 95, to thereby have the clamp assembly 91 squeeze and hold the trunk fiber optic 45 in place. In the presently illustrated embodiment, the set screw 97 is used to hold the chuck 23 in place and prevent rotation thereof. In the illustrated embodiment, the outer surface of the clamp assembly 91 is provided with threads 99 for engaging with corresponding threads on the inner surface of the chuck 23. In the illustrated embodiment, the chuck 23 is screwed onto the threads of the clamp assembly 91, before the removable trunk fiber assembly 16 is inserted into the handpiece 12. The chuck 23 is screwed onto the clamp assembly 91 to a predetermined tightness, and then the set screw 97 is secured thereto to securely hold the chuck 23 to the clamp assembly 91. Subsequently, the removable trunk fiber assembly 16 is inserted and secured into the handpiece head 12.

Referring to FIGS. 5 and 7-9, the fiber tip fluid output device 14 comprises a generally cylindrical body having an outer surface, a proximal end, a distal end, and a lumen extending between the proximal end and the distal end. The lumen is sized and shaped to accommodate the fiber tip 51a therethrough so that the fiber tip 51a extends through the lumen from the proximal end to the distal end of the generally cylindrical body. The fiber tip fluid output device 14 further comprises a plurality of apertures 125 extending around the generally cylindrical body. Each of the apertures 125 fluidly connects the outer surface to the lumen. As presently embodied, the lumen comprises a first diameter near the proximal end and a second diameter near the distal end, wherein in the illustrated embodiment the second diameter is greater than or equal to about two times the first diameter. As presently embodied, the lumen comprises a proximal lumen section and a distal lumen section, the proximal lumen section having a diameter which in the illustrated embodiment is equal to the first diameter and the distal lumen section having a diameter which in the illustrated embodiment is equal to the second diameter. The proximal lumen section comprises a proximal end, a distal end, and a lumen axis extending between the proximal end and the distal end; the distal lumen section comprises a proximal end, a distal end, and a lumen axis extending between the proximal end and the distal end; and the diameter of the proximal lumen section in the illustrated embodiment can be substantially constant along a length of the proximal lumen section between the proximal end of the proximal lumen section and the distal end of the proximal lumen section. The diameter of the distal lumen section can be substantially constant along a length of the distal lumen section between the proximal end of the distal lumen section and the distal end of the distal lumen section. In the illustrated embodiment, the first diameter transitions to the second diameter at the distal end of the proximal lumen section and the proximal end of the distal lumen section, a distal opening of the fiber tip fluid output device 14 has a diameter which is equal to the second diameter, and a proximal opening of the fiber tip fluid output device 14 has a diameter which is equal to the first diameter. In the illustrated embodiment, each of the apertures 125 has a diameter which is about half of the first diameter.

The apertures 125 can be disposed within a first depression 121. A second depression extends around the generally cylindrical body near the proximal end, and a third depression extends around the generally cylindrical body near the distal end, wherein the first depression is disposed about half way between the second depression and the third depression in the illustrated embodiment. As presently embodied, the distal lumen section tapers into the proximal lumen section along a length of the lumen that in the illustrated embodiment is equal to about one third of at least one of the cross-sectional diameters of the apertures 125.

The rotating handpiece 10 of the illustrated embodiment can use the electromagnetically induced cutting system disclosed in U.S. Pat. No. 5,741,247, the entire contents of which are expressly incorporated herein by reference. For example, an engineered and controllable atomized distribution of fluid particles is placed into an interaction for absorption of electromagnetic radiation (from the fiber tip 51a) and for subsequent expansion to impart mechanical cutting forces onto a target or treatment surface. In the illustrated embodiment of FIG. 1, separate air and fluid lines 111, 113, which may be similar to those described in U.S. Pat. No. 5,741,247, run parallel to one another in the distal direction toward the feed channels 115, 117. In other embodiments, the air and fluid lines 111, 113 may comprise a first fluid line for carrying a first fluid and a second fluid line for carrying a second fluid, and further may comprise one or more additional fluid lines (not shown). Thus, while the illustrated embodiment describes the first fluid being air and the second fluid being water, the present disclosure is not limited to such structure and use. For example, the first and second fluids, and additional fluids, may comprise any of the components described in U.S. Pat. No. 5,785,521, the entire contents of which are expressly incorporated herein by reference. Some or all of the components of U.S. Pat. No. 5,785,521 may be premixed and carried through fluid lines, such as the lines 115, 117, or not premixed and mixed within the circumferential chamber 119 discussed below. The feed channels 115, 117, carrying a supply of air and water, respectively, as presently embodied, feed into circumferential chamber 119. Referring to FIGS. 5a-5c, the circumferential chamber 119 can be formed in a first depression 121 of the fiber tip ferrule 123. In an alternative embodiment, the section 121 may not have any depression.

As can be seen from FIG. 5b, for example, four apertures 125 are disposed in the first depression 121 of the fiber tip ferrule 123. In modified embodiments, other numbers of apertures may be incorporated. Air traveling into the circumferential chamber 119 from the feed channel 115, and water traveling into the circumferential chamber 119 from the feed channel 117, are both initially mixed in the circumferential chamber 119. In one embodiment, the first and second fluids may comprise air and a medicated or flavored water, and in another embodiment the first and second fluids may comprise water and at least one other fluid. In still another embodiment, at least one of the first and second fluids may comprise a medicament, such as chlorhexidine gluconate.

The initially-mixed air and water travel from the circumferential chamber 119 through the orifices 125 and into the lumen 133. The air and water is further mixed and atomized within the lumen 133. The atomized water under air pressure subsequently travels along the fiber tip 51 in a direction toward the output end 136 of the fiber tip 51. In a typical embodiment, the fiber tip 51a is permanently affixed to and extends through the fiber tip fluid output device 14. As presently embodied, three O-ring seals 139 are provided to seal the inside of the rotating handpiece from the air and water.

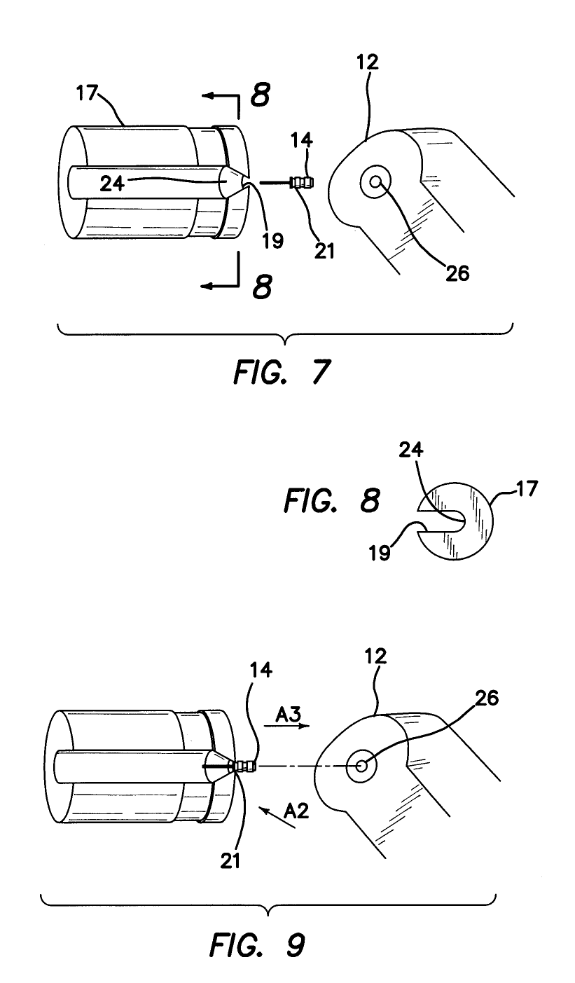

FIG. 7 illustrates the loading tool 17, the fiber tip fluid output device 14, and handpiece head 12 in a disassembled configuration, and FIG. 8 is an end view of the loading tool 17, taken along the line 8-8 of FIG. 7.

FIG. 9 shows the fiber tip fluid output device 14 partially secured onto the loading tool 17. The proximal end of fiber tip fluid output device 14 can be gripped by the hand of a user and slid into the slot 19 of the loading tool 17 in the direction of the arrow A2. As presently embodied slot 19 fits around the third depression 21 of the fiber tip fluid output device 14, and the fiber tip fluid output device 14 is slid within the slot 19 in the direction of the arrow A2 until the fiber tip fluid output device 14 reaches the end 24 of the slot 19. The loading tool is then advanced in the direction of the arrow A3 to firmly secure the fiber tip fluid output device 14 into the orifice 26 of the handpiece head 12. The loading tool 17 is then removed from the fiber tip fluid output device 14 to leave the fiber tip fluid output device 14 firmly secured within the orifice 26. As presently embodied, a width of the slot 19 is slightly larger than a diameter of the third depression 21, so that the fiber tip fluid output device 21 can be removably and snugly held by the loading tool 17.

Referring to FIG. 3, the removable trunk fiber assembly 16 can be provided with three radial ports for introducing air, water, and (optionally) cooling air. More particularly, a fluid radial channel 161 feeds fluid (e.g., water) into the fluid channel 111, an air radial channel 163 feeds air into the air channel 113, and an optional cooling-air radial channel 165 feeds cooling air along a cooling-air channel, which exits in close proximity to the parabolic mirror 41. In a representative embodiment, the exit angle of the cooling air channel directs cooling air directly onto the parabolic mirror 41, so that the cooling air is reflected from the parabolic mirror 41 onto the input end 59 of the fiber tip 51 and, subsequently, onto the window 43. In FIG. 2, the cooling air exits from an orifice 181a and is channeled directly onto the input end 59a of the fiber tip 51a. Subsequently, the air is directed onto the parabolic mirror 41 and reflected onto the output end 55 of the trunk fiber optic 45. This configuration could also be implemented for the system of FIG. 1, wherein the cooling air subsequently is directed onto the window 43. Alternatively, in the embodiment of FIG. 2, the cooling air exiting the orifice 181a can be channeled directly onto the parabolic mirror 41, focusing onto the input end 59a of the fiber tip 51. In the embodiments of both FIG. 1 and FIG. 2, the cooling air is subsequently channeled in the direction of the arrows A2 through channels formed in the chuck 23. As shown in FIG. 3a, the chuck 23 can have portions of its two sides removed, to thereby form channels for passage of the cooling air. The cooling air travels through the channels of the chuck 23 under a vacuum pressure and, subsequently, is drawn into a removal port 191. Upon entering the removal port 191 under the vacuum, the cooling air travels in a direction opposite to the arrow A1 and exits the removal trunk fiber assembly 16. The four O-rings 196 insulate the radial channels 161, 163, 165 from one another.

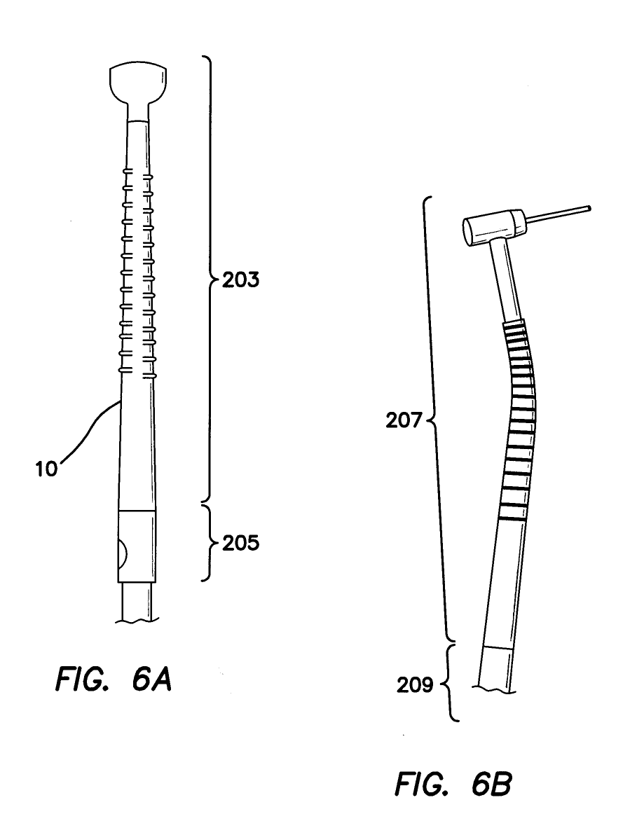

FIG. 6a illustrates a side elevation view of the assembled rotating handpiece 10 and FIG. 6b illustrates a modified embodiment of the rotating handpiece 10, wherein the neck is slightly bent. In FIG. 6a the portion indicated by reference numeral 203 is adapted to rotate about an axis of the rotating handpiece 10. The portion 205 does not rotate. Similarly, in FIG. 6b, the portion 207 is adapted to rotate about an axis of the rotating handpiece, and the portion 209 docs not rotate. In the embodiment of FIG. 6b, the trunk fiber optic is configured to be slightly flexible, since the trunk fiber optic will need to bend and flex as the portion 207 is rotated relative to the portion 209. In either of the embodiments of FIGS. 6a and 6b, the user holds the rotating portion (203 or 207) with his or her thumb and two fingers (such as is conventional in the art) and allows the stationary portion (205 or 209) to rest on a portion of the hand bridging the user's forefinger and thumb. The three fingers holding the rotating portion (203 or 207) contact the rotating portion and can rotate the rotating portion, as the fixed portion (205 or 209) does not rotate and rests on the portion of the hand bridging the hand and the forefinger.

The following figures show exemplary embodiments of radiation emitting apparatuses which are constructed to emit electromagnetic radiation in non-centered or non-concentrically focused manners, relative to the output from a cylindrically-shaped fiber optic end (i.e., a truncated fiber end), onto target surfaces or treatment sites. The target surface or treatment site can comprise, for example, a part of the body, such as a tooth, a knee, a wrist, or a portion of the jaw to be treated.

The output radiation can be engineered to have a spatial energy distribution which differs from the spatial energy distribution of a conventional truncated fiber end. More particularly, in accordance with an aspect of the present invention, a radiation emitting apparatus is constructed to generate output radiation having a spatial energy distribution with one or more energy concentrations or peaks located in areas other than a center of the spatial energy distribution. The center of the spatial energy distribution can be defined as an area aligned with (or intersecting) an optical fiber axis of the shaped fiber optic tip or an area aligned with (or intersecting) an average direction of propagation of the output radiation. According to one aspect, the center of the spatial energy distribution can be defined as a central part of a cross-section of the output radiation taken in a direction orthogonal to the direction of propagation of the output radiation.

In accordance with an aspect of the present invention, the side-firing output ends described herein may be used for caries removal from predetermined locations (e.g., side walls) of tooth cavities. Using the side-firing output ends of the present invention, undercuts may be effectively generated in caries procedures wherein each undercut may comprise a removed volume of caries defining a reverse-mushroom shaped aperture in the tooth which has a size at the surface of the tooth that is less than sizes of the aperture beneath the surface and which is to be filled with amalgam. Sizes of the aperture of such an undercut may progressively increase with distance away from the tooth surface in a direction toward a center of the tooth. For example, a dentist may insert a curved stainless steel probe into a cavity, detect caries material on a surface (e.g., sidewall) of the cavity, remove the curved stainless steel probe, insert a shaped fiber optic tip of the present invention having a side-firing output end into the cavity, position the side-firing output end to ablate the detected caries material, activate a laser to remove the detected caries material, and then (optionally) repeat the process until all detectable or a desired level of caries material has been removed. The shaped fiber optic tips of the present invention, and in particular their side-firing output ends, can thus facilitate generation of reverse-mushroom shaped apertures by way of operation of their side-firing characteristics, which can facilitate, for example, removal of tissue (e.g., caries) from side walls of the cavity down beneath the surface of the tooth.

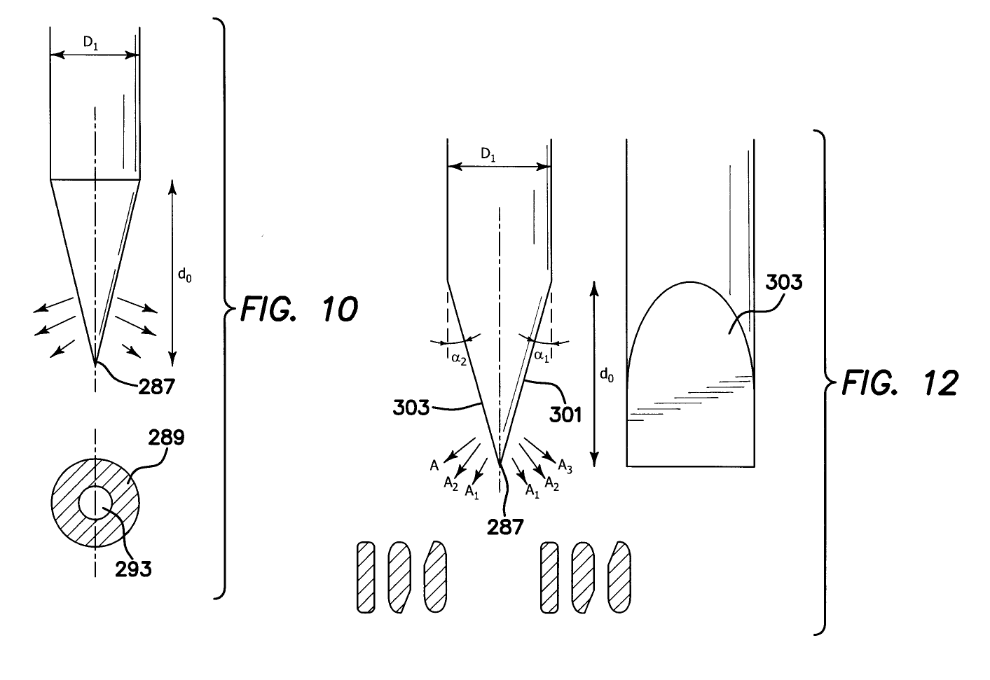

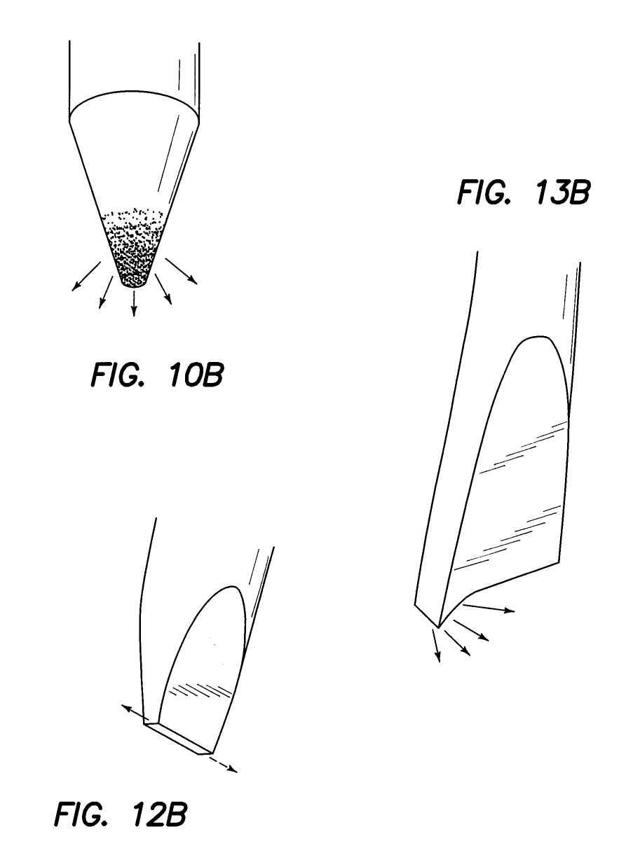

With particular reference to FIG. 10, a cross-sectional view of a shaped fiber optic tip is illustrated. The shaped fiber optic tip comprises a cylindrically-shaped body having a diameter D1 of about 200 to about 2000 microns, such as, for example, 1200 microns, and further comprises a distal, conically-shaped, side-firing output end. The side-firing output end in the depiction generates a pattern having a ring pattern 289 which surrounds a non-illuminated central area 293.

In accordance with another aspect of the present invention, dimensions of the side-firing output ends of the shaped fiber optic tips can be selected to obtain internal reflection within the shaped fiber optic tip at, for example, the tip/air interface, as elucidated for example in FIG. 11. In particular implementations, dimensions of the side-firing output ends of the shaped fiber optic tips can be selected to obtain total, substantial or a large degree of internal reflection of electromagnetic radiation at one side (e.g., side 303 and/or side 317) and firing through another (e.g., the opposite) bevel-cut side (e.g., side 301) of the side-firing output end of the shaped fiber optic tip.

With reference to FIG. 11, in an exemplary context of a side-firing output end, the full angle (i.e., angle between surfaces forming the angle) at a distal region of the side-firing output end can be in the range from 10 degrees to 170 degrees, and in particular examples between 50 degrees and 100 degrees. Generally, the shaped fiber optic tip can have a diameter between about 200 and about 2000 microns, and can have a numerical aperture (N.A.) depending on the material. The exemplary shaped fiber optic tip can be made of silica or other materials, such as sapphire, or other materials disclosed in U.S. Pat. No. 5,741,247, the entire contents of which are incorporate by reference herein, and can also comprise a hollow waveguide in modified embodiments.

In particular implementations, any of the structures disclosed herein may be formed of quartz (e.g., low or high OH), sapphire, glass, or similar material. According to any of the embodiments disclosed herein, the shaped fiber optics may be straight or bent at an angle (90°, 60°, 45°, 30°, 15°) (e.g., 10 mm from distal end), wherein one or more of the bend radius, bend angle, and location of the bend (e.g., from the distal end) can be adjusted depending on the nature and the location of the procedure to be performed. The shaped fiber optics may be used, for example, in cannulas for operating inside channels (e.g. blood vessels) or deep pockets.

In the exemplary embodiment of FIG. 11, the shaped fiber optic tip comprises a 600 micron core diameter, a numerical aperture of 0.39, an acceptance angle, α₁, of 15.6 degrees, and a full angle of 59 degrees to 62 degrees.

The full angle can be determined using, for example, Snell's Law of Refraction, nₒ sin(αₒ)=n₁ sin(α₁), for all waveguide modes to experience total internal reflection on at least one of the tapered surfaces of the side-firing output end before exiting through the side-firing output end. More particularly, in the illustration of FIG. 11, the structure comprises a first tapered surface (shown near top of drawing page) and an opposing second tapered surface (shown near bottom of drawing page). According to an implementation of the present invention in which internal reflection occurs, light striking the first tapered surface is reflected toward and exits through the second tapered surface to thereby achieve a side-firing effect.

In the illustration, the refractive indices nₒ and n₁ can be 1.0 and 1.45, respectively, corresponding to an implementation of a quartz side-firing output end transmitting into air, and further values may be implemented wherein αₒ=8.0 degrees and α₁=5.5 degrees. Beginning with an equation that (½)α_{cone}+α₁+α_{t.r.}=90 degrees, wherein α_{cone} is defined as the total angle and α_{t.r.} is defined as the angle for total internal reflection, the angle for total internal reflection, α_{t.r.}, can be isolated to yield α_{t.r.}=sin⁻¹(nₒ/n₁) which in the present example equals 43.6 degrees. When (½)α_{cone}=40.9 degrees, the total angle can be determined in the example as α_{cone}=81.8 degrees.

Although the full angle in the illustrated embodiment is selected to facilitate a large degree, or total, internal reflection, modified embodiments (e.g., having other shapes or materials) or other side-firing output ends may be constructed wherein the internal reflection (i.e., reflection off of a first surface or first tapered surface, or the percentage of reflection from light first striking any tapered or other surface of the side-firing output end) can be about 50% or greater. In still other embodiments, a total angle can be constructed to provide for an internal reflection of at least 25%. In further embodiments, however, other varying amounts of internal reflection can be implemented.

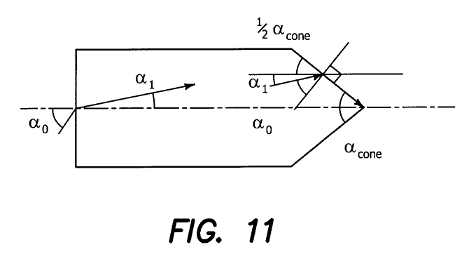

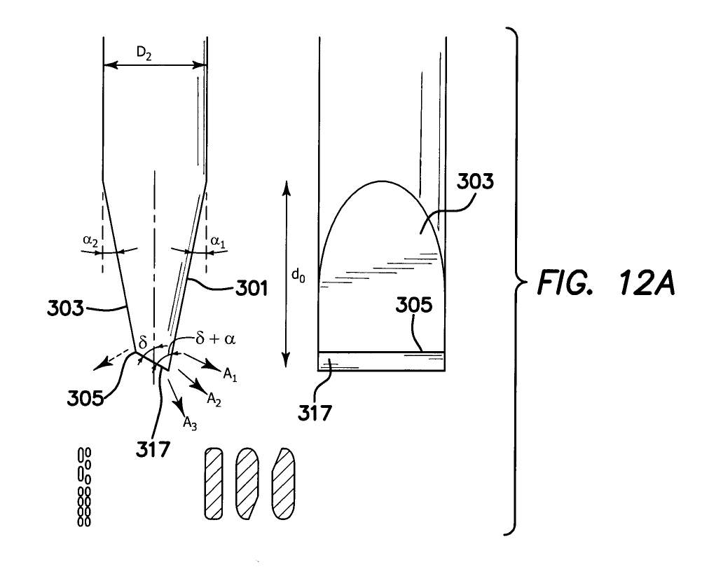

In FIGS. 12A and 12B, a plurality (e.g., two) members 301 and 303 are formed (e.g., beveled) on a side-firing output end of a shaped fiber optic tip. The members in typical implementations comprise sides, such as sides (e.g., planar surfaces) 301 and 303, that taper in an output direction of propagation of electromagnetic radiation. Additionally, in typical implementations, the sides 301 and 303 are formed on (e.g., on opposing sides of) a cylindrically shaped body of a fiber optic to yield a shaped fiber optic tip with a side-firing output end that generates a side-firing irradiation pattern, as exemplified in the figure with arrows A1, A2 and A3. The dimension d₀ is provided for reference in both FIGS. 10 and 11, and in subsequent figures.

FIG. 12 and other figures which follow comprise sides 301 and 303, which, when generated, form angles with respect to optical axes of the shaped fiber optic tips. The side 301 is generated to form an angle α₁ with respect to the optical axis of the shaped fiber optic tip and the side 302 is generated to form an angle α₂ with respect to the optical axis of the shaped fiber optic tip; and, as presently embodied, the angle α₁ can range from about 1 to about 10 degrees, the angle α₂ can also range from about 1 to about 10 degrees, and the diameter D2 can range from about 200 to about 2000 microns. The structure in the depiction on the right-hand side of FIG. 12 is rotated 90 degrees with respect to the left-hand depiction of the same figure. The shaped fiber optic tip may comprise, for example, sapphire, diamond, or quartz (glass). In certain embodiments, portions of the presently discussed tip, and of any other tip disclosed herein, may be “frosted” to provide for different types of light diffusion (e.g., scattering) effects.

According to typical embodiments, each side of the side-firing output end is formed by polishing. For example, the shaped fiber optic tip may be grasped and moved to position a distal end thereof onto an operative surface of a polishing machine. The distal end of the shaped fiber optic is then oriented with respect to the operative surface, and held (e.g., at a steady orientation) to remove portions of the fiber and to form a beveled surface (e.g., surface 301) onto the side-firing output end of the shaped fiber optic tip. Subsequently, the shaped fiber optic tip may be moved to position an opposite distal end thereof onto the operative surface of the polishing machine to remove portions of the fiber and to form another beveled surface (e.g., surface 303) onto the side-firing output end of the shaped fiber optic tip.

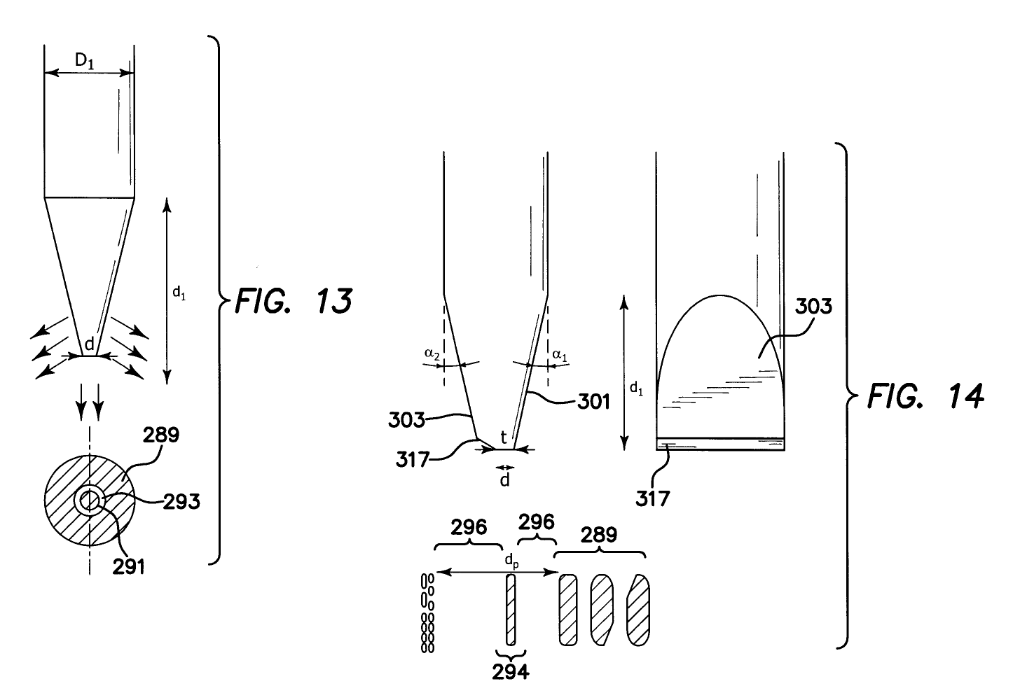

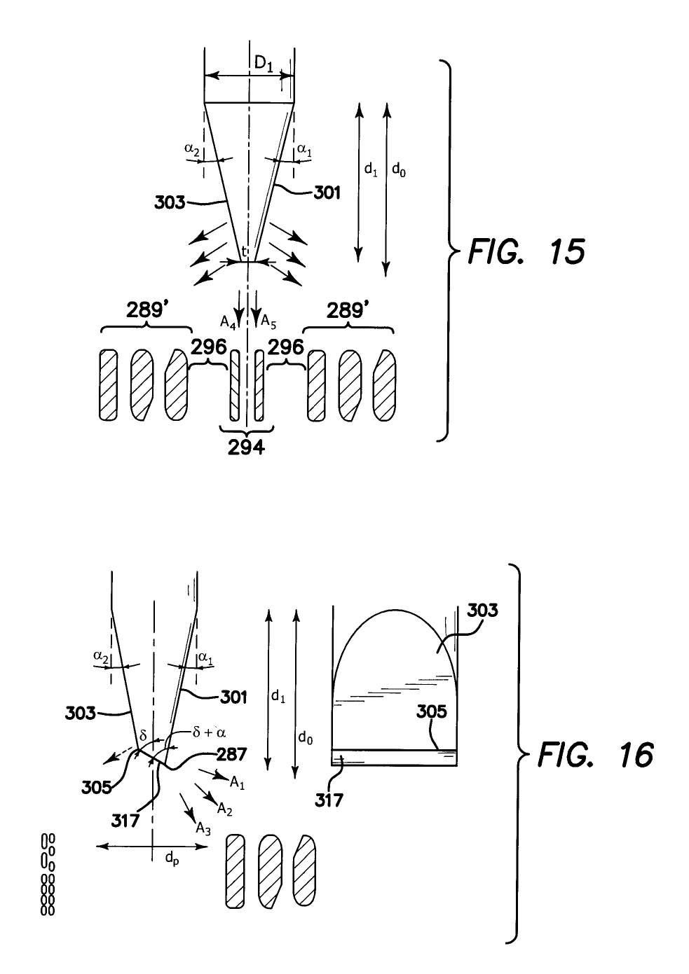

As for the formation of the two sides 301 and/or 303, they may be polished all of the way down to form a distal edge 287, or may be polished a lesser amount at the far distal end to form a truncated distal surface having a thickness t such as depicted in FIG. 15. The thickness t is provided in figures other than FIG. 15, such as FIGS. 14 and 29, for reference.

In another implementation, the two sides 301 and/or 303 may be full or partially formed by polishing, followed by polishing of the distal end of the structure, followed by polishing of the two sides 301 and/or 303, followed by additional polishing of the distal end to generate the truncated distal surface. Polishing of the sides and/or subsequent polishing after formation of the sides, to yield a structure having a truncated distal surface can result in irradiation, exemplified with arrows A4 and A5, exiting through the truncated distal surface to form an irradiation pattern having an illuminated area 294.

Regarding FIG. 15 and its truncated distal surface configuration, generally, the constructions of FIGS. 13, 14, 15, 28, 29, 31, 32A, 32B, 33A and 33B correspond to a shaped fiber optic tip with a truncated (e.g., flattened or, in other implementations, not sharp) distal surface. For example, part of the distal end and/or side 303 and, optionally, side 301, can be polished to form a surface at the far distal end that is normal (e.g., or substantially normal) to the optical axis of the shaped fiber optic) to form a truncated (e.g., flattened or, in other implementations, not sharp) distal end. The forming of a side 317 on a structure having a truncated (e.g., flattened or, in other implementations, not sharp) distal surface, can be implemented to generate a distal edge 287 or to not form a distal edge. In a modified embodiment, a side 317 may be formed on a structure having a truncated (e.g., flattened or, in other implementations, not sharp) distal surface, to generate a distal edge. Furthermore, in certain implementations, following formation of the side 317, the structure can be subsequently modified.

Turning from FIG. 12 to FIGS. 12A and 12B, these figures provide views of a side firing tip comprising a shaped fiber optic tip having dual bevel-cut side-firing output ends according to an embodiment of the present invention, wherein sides (e.g., bevel cuts) 301 and 303 taper in an output direction of propagation of electromagnetic radiation. The structure in the depiction on the right-hand side of FIG. 12A is rotated 90 degrees with respect to the left-hand depiction of the same figure. In a typical embodiment, as before, the side-firing output ends can comprises a material such as sapphire, diamond or quartz that is polished to a bevel-cut shape.

In accordance with an aspect of the present invention, in addition to sides 301 and 303, an additional member (e.g., side 317) is embodied to facilitate or alter at least one characteristic of internal reflection within, or an output from, the side-firing output end. For procedures that require removal of tissue from one surface only so that other surfaces are not affected, the shaped fiber optic tip of the current invention can emit energy only toward the one surface that needs to be treated. The shaped fiber optic tip, according to an aspect of the invention, can be designed to maximize the area of the output energy emission (e.g., off-axis emission) by increasing the surface that allows for total reflection of laser energy (cf. drawings and the different angles for the total reflection surface). For example, side 317, as depicted on the left-hand side of FIG. 12A, can be formed to reflect distally-directed electromagnetic energy to side 301. The arrows A1, A2 and A3, and the corresponding irradiation patterns, in FIG. 12A, indicate such electromagnetic radiation being internally reflected via side 317 and exiting from side 301 of the side-firing output end to generate an off-axis (e.g., side firing) irradiation pattern on a target.

To construct the shaped fiber optic tip with, for example, an additional member, in connection with the currently described or any of the preceding or following embodiments, the shaped fiber optic tip may be grasped and moved to position a distal end thereof onto an operative surface of a polishing machine, such as a machine as mentioned previously, with the distal end of the shaped fiber optic being oriented with respect to the operative surface, and not rotated, to remove portions of and polish a side 301 of the distal end of the shaped fiber optic tip into a bevel-cut side-firing output end. The side 301 can be formed, for example, to have an angle α₁, measured between the optical axis of the fiber optic tip and the surface of side 301, ranging from about 1 to about 10 degrees. The shaped fiber optic tip then may, optionally, be rotated 180 degrees, or another angle in modified embodiments, and the procedure can be repeated, in whole, in part, to the same, to a greater, or to a lesser degree, to remove the same, similar, or dissimilar portions of and polish a side 303 of the distal end of the shaped fiber optic tip, thereby yielding a structure with two output-modified (e.g., flattened) sides 301 and 303 that taper to a point (e.g., truncated or not truncated). The side 303 can be formed, for example, to have an angle α₃, measured between the optical axis of the fiber optic tip and the surface of side 303, ranging from about 1 to about 10 degrees. Subsequently, the distal end of the shaped fiber optic be can oriented with respect to the operative surface, to remove portions of side 303 of the shaped fiber optic tip for formation of a side 317. The side 317 may be formed by holding the shaped fiber optic tip (e.g., at a steady orientation) to remove portions of the shaped fiber optic and to form by polishing the side 317. The side 301 can be formed, for example, so that the angle α₁, measured between the optical axis of the fiber optic tip and the surface of side 301, ranges from about 20 to about 80 degrees. Alternatively, the distal end of the shaped fiber optic can oriented with respect to the operative surface, to remove portions of one or more of side 303 and a distal end of the shaped fiber optic tip. In other embodiments, the distal end of the shaped fiber optic can oriented with respect to the operative surface, to remove portions of one or more of side 303, a distal end of the shaped fiber optic tip, and side 301, and, optionally, of any other side, and, optionally, the process repeated in any combination any additional number of times.

The side 317 may be generated at an orientation to form an angle δ with an optical axis of the side-firing output end, wherein, in accordance with certain aspects and implementations, larger angles δ may facilitate greater amounts of one or more of internal reflection, output through side 301 and a more dispersed output of electromagnetic energy from side 301. On the other hand, in accordance with other aspects and implementations, some angles δ may be so small that undesirable amounts of electromagnetic radiation exit distally and/or an insufficient amount of light exits from side 301. The side 317 may be generated at an orientation to form an angle δ with an optical axis of the side-firing output end, wherein, in accordance with an aspect of the invention, the angle δ can range from about 30 degrees to about 90 degrees. The side 317 may be generated at an orientation to form an angle δ with an optical axis of the side-firing output end, wherein, in accordance with certain embodiments, the angle δ can range from about 45 degrees to about 60 degrees. In a particular implementation, the angle δ can be about 57 degrees.

Characteristics of the sides (e.g., sides 301, 303 and/or 317), such as the angles α₁, α₃ and/or δ, do not need to be the same or even similar. For instance, the sides (e.g., sides 301 and 303) may be oppositely disposed or disposed at non-opposing positions (e.g., at angles other than 180 degrees), and/or may number in two as illustrated or fewer or greater, and/or may be worked to the same shape and/or the same amount removed as illustrated and/or different shapes or amounts removed. In the illustrated example of FIG. 12A, the sides 301 and 303 are similar. In certain embodiments, the resulting shape, or blade, as illustrated on the left-hand side of FIG. 12A, may be compared to the shape of a flat-head screw driver.

In accordance with an aspect of the present invention, the distal ends (e.g., blades) of the side-firing output ends described herein may be used for removing calculus deposits from tooth surfaces. For instance, the distal ends (e.g., blades) may exert (e.g., focus) mechanical removal forces onto the calculus deposits in combination with the electromagnetic energy applying other (e.g., laser-related rather than blade-related) removal forces, such as described and referenced herein including mechanical forces and/or disruptive removal forces, to the calculus deposits.

According to an aspect of the present invention, shaped fiber optic tips (e.g., fiber optics) are provided which are capable of emitting large cross-sectional areas (e.g., distributions) of energy to remove tissue and deposits or lesions present on soft or hard tissues (e.g., calculus from the root or the tooth surface, soft and calcified plaque from teeth or from within blood vessels, surface lesions, surface stains, etc.). According to an exemplary method of the present invention, a side-firing output tip can be applied to a calculus deposit (e.g., with the blade facing the deposit at an angle and oriented so that electromagnetic energy and/or fluid particles and/or disruptive forces as described or referenced herein or as inherent from the current disclosure is/are emitted into the calculus deposit) and moved back and forth over the deposit (e.g., in a direction such that the blade is oriented in a direction transverse to the forward and backward movement of the side-firing output tip (similar to the alignment and motion used to remove or clean a surface with a scraper) over the calculus deposit. Laser energy (e.g., pulsed laser energy as described or referenced herein) can be applied in one or more of (a) forward and (b) backward movements of the side-firing output tip. The above features can be applied in various combinations to facilitate, for example, removal of the calculus deposit wherein the calculus deposit is removed, in a typical embodiment, in layers corresponding to the forward and backward movements of the side-firing output tip.

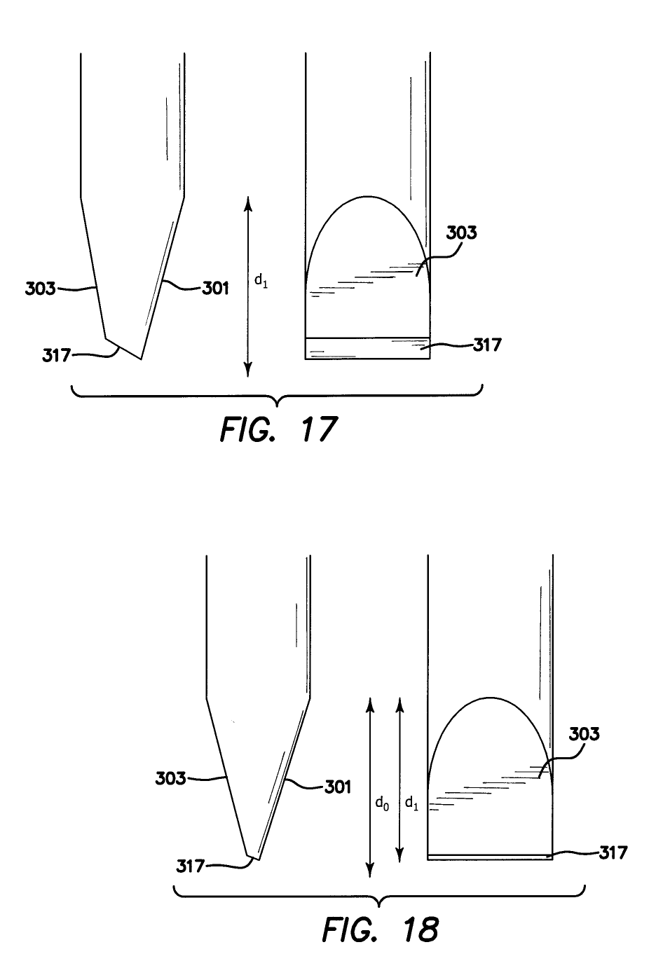

According to a number of implementations, a side 317 may be formed on a structure having a distal edge 287 (cf. FIG. 12), either to keep the same edge 287 (cf. FIG. 12A wherein the distal tip is edge 287) or to form another (e.g., an alternative) distal edge (cf. FIG. 17 with implementations of surface 317 being formed at, for example, the same orientation but further proximally so that a new distal edge is generated). Such a side 317 may, for instance, be polished so much as to shorten a length of the side-firing output end. In such or alternative implementations, following formation of the side 317, the structure may be subsequently modified (e.g., polished to form a surface at the far distal end that is normal to the optical axis of the shaped fiber optic) to form a truncated (e.g., flattened or, in other implementations, not sharp) distal end.

When a side 317 is polished so much as to shorten a length of the side-firing output end one or more output characteristics of the side-firing output end may be altered. An effect which may result from a shortened side-firing output end may comprise, for example, a wider irradiation pattern, as elucidated, for example in FIG. 16. Comparing the irradiation pattern of FIG. 16 to those of FIGS. 14 and 15, in which the side-firing output end is not shortened from the generating of side 317, the length of the arrow dₚ in FIG. 16 symbolizes the length of the irradiation pattern formed by the structures of FIGS. 14 and 15. Now with reference to FIG. 16, it can be discerned that the irradiation pattern of FIG. 16 is greater than the dimension dₚ.

Another effect that may occur during or as a consequence of the formation of the side 317 may comprise, for example, a new distal edge in a different location and/or orientation as compared to, for example, a location/orientation of one or more of (a) the optical axis of the side-firing output end, (b) a prior distal edge, and (c) a prior truncated distal surface, which existed prior to formation of the side 317.

With regard to FIG. 26, formation of the side 317 (e.g., from a structure not having a truncated distal surface) results in shortening of the side-firing output end and, further, results in a new, distal edge that is in a different location with respect to the optical axis (shown in phantom) of the side-firing output end.

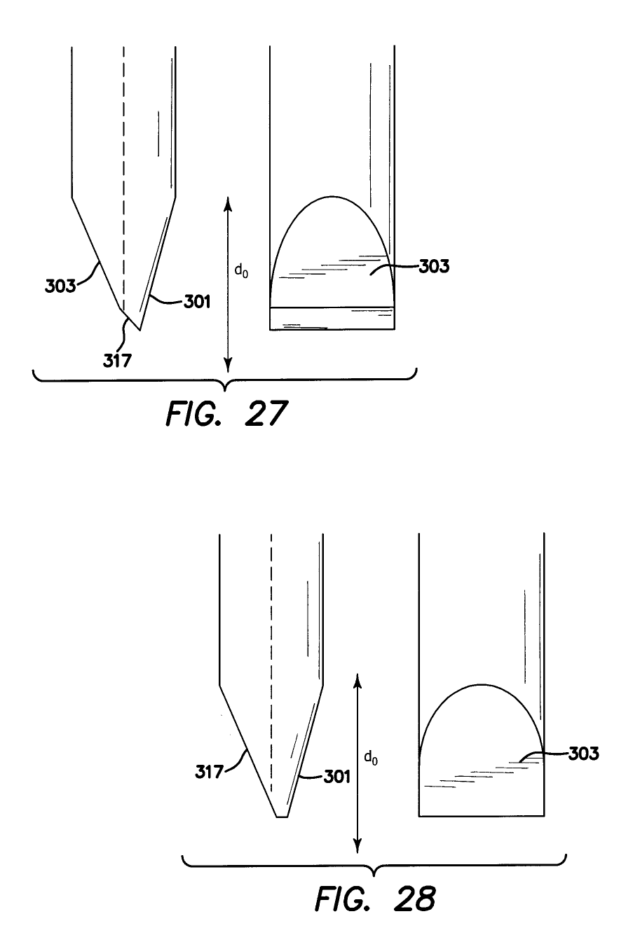

In FIG. 27, formation of a side, such as, for example, the side depicted in FIG. 26 with reference designator number 317, may be performed to yield a side 303 in FIG. 27. Alternatively, the side 303 may be formed initially along with side 301. The sides 301 and 303, in the illustrated embodiment, form different angles with respect to the optical axis (shown in phantom). When side 317 is then formed, shortening (e.g., further shortening) of the side-firing output end occurs.

A side 317 is formed in FIG. 28, by way of, for example, either the process described in connection with FIG. 26 or an initial process along with formation of side 301 (e.g., so that, in the latter scenario, side 317 is formed initially without a side 303 ever being formed). The sides 301 and 317 form different angles with respect to the optical axis (shown in phantom). According to the latter scenario, formation of side 317 results in shortening of the side-firing output end. A truncated distal surface can, optionally, be formed on the resulting structure.

In accordance with an aspect of the present invention, in addition to members (e.g., sides) 301, 303, and 317, a further member (e.g., side) is embodied to facilitate or alter at least one characteristic of internal reflection within, or an output from, the side-firing output end. Typically, a further side is formed to reflect distally-directed electromagnetic energy to side 301.

The structure of FIG. 29 may be generated from, for example, a structure such as depicted in FIG. 15. In one implementation, following formation of the FIG. 15 structure, a first side (cf. 317) may be formed on side 303 for directing electromagnetic energy to side 301 and, subsequently, another such side may be formed on one or more of the side 303 and the first side. In a typical implementation, such as that depicted in FIG. 29, the other side can be formed between the distal end (e.g., the truncated distal surface) of the side-firing output end and the first side. For example, the structure of FIG. 29 may be formed from a structure such as depicted in FIG. 15, having a truncated distal surface of thickness t, wherein the thickness t is preserved and wherein the other side is disposed between the truncated distal surface and the first side. In a modified implementation, the structure of FIG. 29 may be formed from a structure as shown in FIG. 15 having a truncated distal surface of thickness t, wherein only part of the thickness t is preserved (e.g., the thickness is less than thickness t) on the truncated distal surface and wherein the other side is disposed between the truncated distal surface and the first side.

The structure of FIG. 30 can be formed, for example, from a structure as depicted in FIG. 15 having a truncated distal surface of thickness t, but can be formed to have a distal edge. A first side (cf. 317) may be formed on side 303 for directing electromagnetic energy to side 301 and, subsequently, another side may be formed on one or more of the side 303 and the first side, wherein the resulting structure comprises a distal edge. The other side may be disposed between the distal edge and the first side. In the implementation of FIG. 30, the thickness t from FIG. 15 is, of course, not preserved and, instead, a distal edge is formed at the distal end of the side-firing output end. Alternatively, the structure of FIG. 30 may be formed from an initial structure not having a truncated distal surface.

Furthermore, according to other aspects of the present invention, in addition to members (e.g., sides) 301, 303, 317, and the other member (e.g., side), one or more further members may be embodied to facilitate or alter at least one characteristic of internal reflection within, or an output from, the side-firing output end.

The structure of FIG. 17 is similar to that of FIG. 14 in that a length of the side-firing output tip has been shortened during formation (e.g., from an initial structure having a truncated distal surface such as shown in FIG. 13) of side 317. A length d₁ of the resulting structure is thus not maintained to be the same as the length d₁ of the initial structure depicted, for example, in FIG. 13. The dimension d₁ is provided for reference in FIGS. 13, 14 and 17, and in prior and subsequent figures.

The structure of FIG. 18 is also similar to that of FIG. 14 to the extent that a length of the side-firing output tip has been shortened during formation of side 317. This structure, however, begins with an initial fiber configuration that does not comprise a truncated distal surface, such as that shown in FIG. 12. As the side 317 is generated, a length d₀ of the resulting structure is not maintained to be the same as the length d₀ of the initial structure depicted, for example, in FIG. 12.

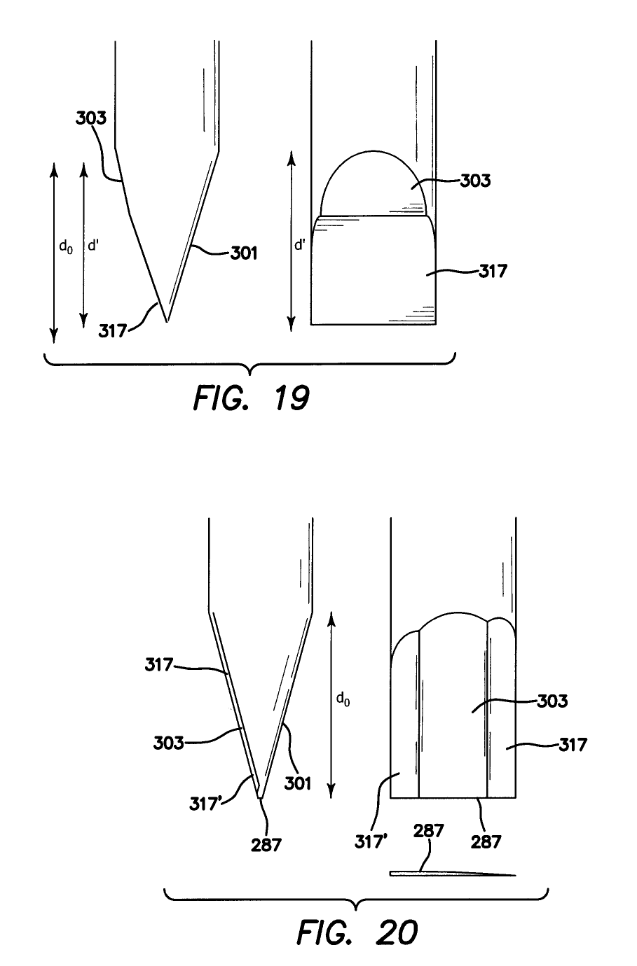

In FIG. 19, side 317 is formed in a fashion similar to that describe with reference to FIG. 18, wherein the length d′ of the resulting structure is less than the length d₀ of the initial structure depicted, for example, in FIG. 12.

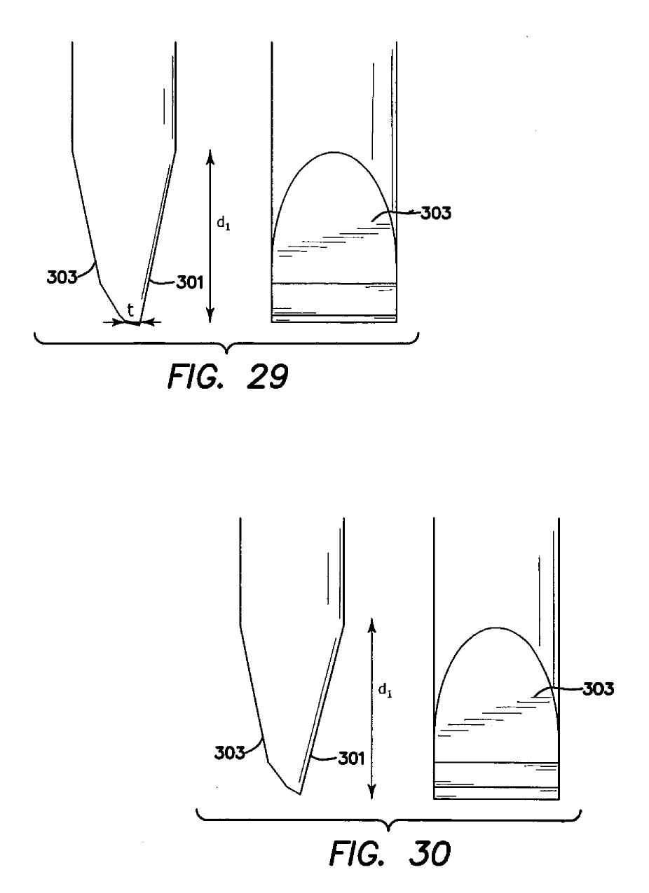

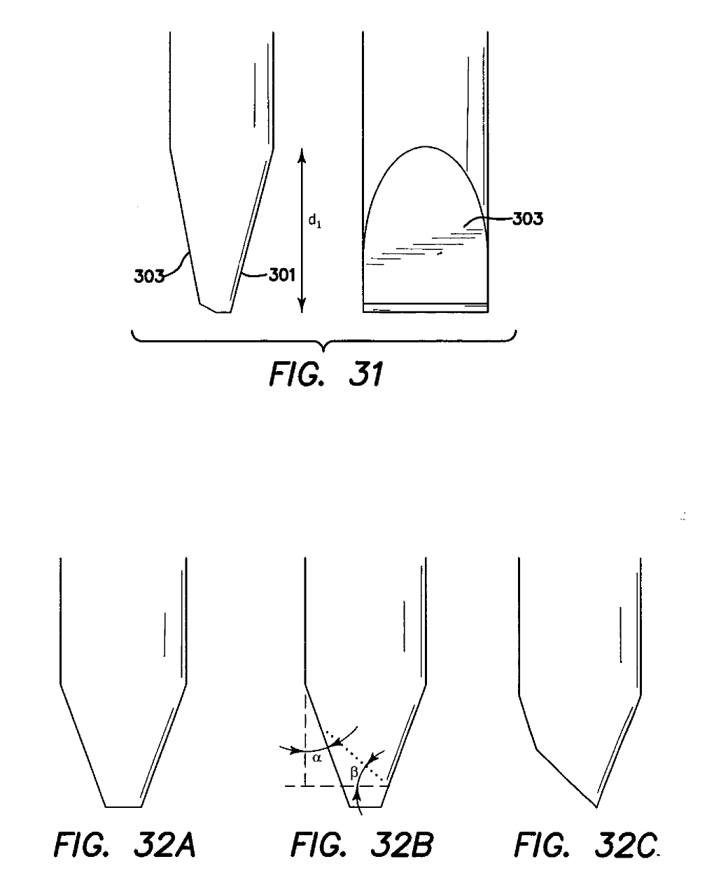

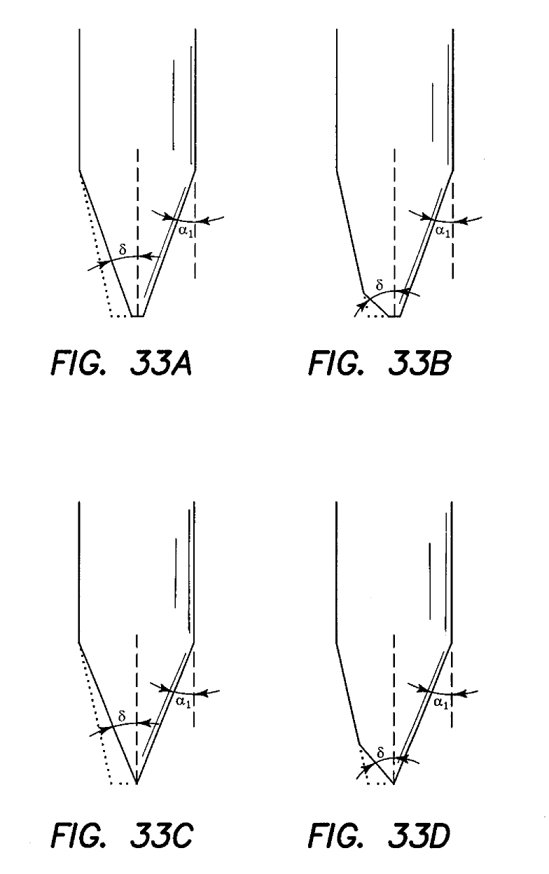

FIGS. 32A-32C illustrate formation of a structure similar, for example, to that depicted in FIG. 17, beginning with a configuration similar to FIG. 15 (cf. FIG. 32A), the analysis of where side 317 will be formed (cf. FIG. 32B), and the formation of the resulting structure (cf. FIG. 32C). FIGS. 33A and 33B illustrate formations of structure similar, for example, to that depicted in FIG. 14, and FIGS. 33C and 33D illustrate formations of structures similar, for example, to that depicted in FIG. 16.

In the constructions of FIGS. 32A-32C and 33A-33D, the angles α, and α₁, corresponding to α₁ and α₂ of other figures provided herein, can be about 1 to about 10 degrees. In typical implementations, the angles α and α₁, and/or one or more of the angles α₁ and α₂, can be about 2.5 to about 3 degrees. Furthermore, the angle β in FIG. 32B, and/or any other structure described herein, can be about 20 to about 80 degrees. In certain implementations, the angle β can be about 45 to about 60 degrees. In a particular embodiment, the angle β can be about 57 degrees. The angle δ in FIGS. 33A-33D, and/or any other structure described herein, can be about 10 to about 70 degrees or, in certain implementations, about 30 to about 45 degrees or, in a particular embodiment, about 33 degrees. As with earlier described implementations, the shaped fiber optic tips depicted in FIGS. 32A-33D can comprise cylindrically-shaped bodies, and diameters of the shaped fiber optic tips can be about 200 to about 2000 microns, such as, for example, 1200 microns. Furthermore, profiles of distal ends (e.g., distal edges 287) of the side-firing output tips, such as that shown in the version A bottom end view depiction of FIG. 22, can comprise thicknesses (e.g., the smaller dimension, corresponding to thickness t of, for example, FIG. 15) of about 300 microns and widths (e.g., the other dimension, corresponding to a diameter of the shaped fiber optic tip) of about 1200 microns.

Typically, the shaped fiber optic tips described herein may be operated at 2-50 mJ/pulse in a contact mode and may be operated at 50-100 mJ/pulse in a non contact mode. The shaped fiber optic tips described herein may be operated, for example, at 50 Hz frequencies and a 20 mJ/pulse parameter to generate a 1 W power output and at a 40 mJ/pulse parameter to generate a 2 W power output. Also, the shaped fiber optic tips may be operated, as other examples, at 70 Hz and 20 mJ/pulse to output 1 W, at 70 Hz and 40 mJ/pulse to output 2 W, at 100 Hz and 20 mJ/pulse to output 1 W and at 100 Hz and 40 mJ/pulse to output 2 W. When used with fluids, as described herein, typical flow rates may comprise 0.01 to about 4 ml/minute and, in particular implementations, flow rates of about 0.8 to about 2 ml/minute.

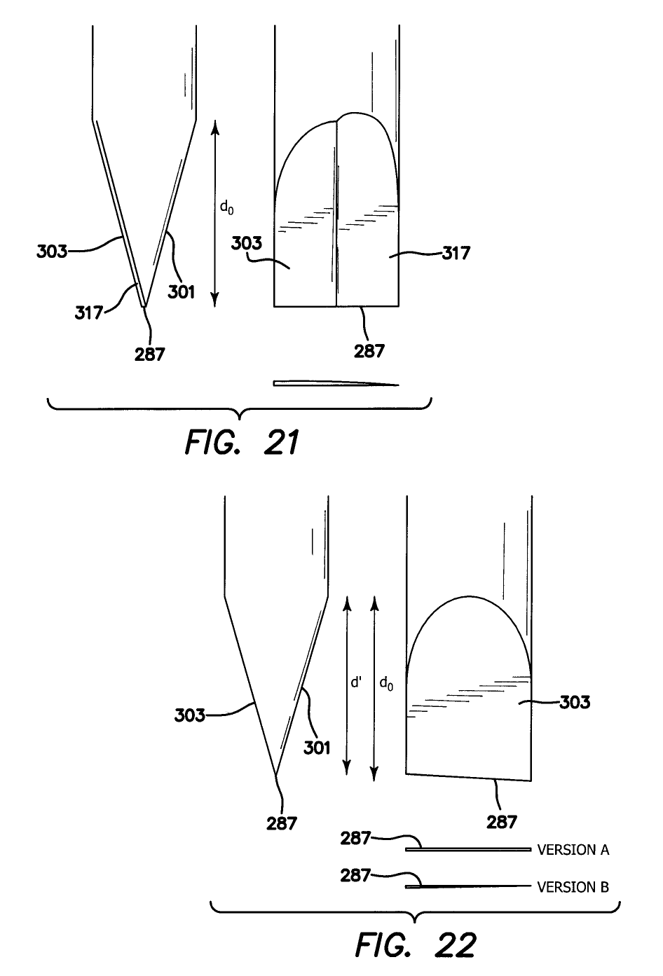

While the depiction of, for example, FIGS. 10, 12A and 12B shows two sides 301 and 303, either of these sides may be formed to comprise similar or substantially different shapes, and, furthermore, additional sides may be formed, as well. As an example, if the polar orientation of side 301 is zero degrees and the polar orientation of side 303 is 180 degrees, then a modified embodiment may comprise side 301 being formed of two planes (rather than just one) having orientations of, for example, zero and one degrees. An intersection of these two sides may be along, for example, a line extending parallel to the optical axis of the shaped fiber optic tip (e.g., so that the side 303 is bifurcated or otherwise divided longitudinally into two planar sides having orientations differing by, for example, one or more degrees). The construction of FIG. 20 shows an implementation wherein two surfaces 317 and 317′ are formed on opposing sides of surface 303. The bottom image in the figure is a bottom end view depicting a profile of the distal edge 287. The construction of FIG. 21 shows an implementation wherein one surface 317 is formed on or next to surface 303. The bottom image in the figure is a bottom end view depicting a profile of the distal edge 287. Alternatively, as another example of many possible implementations, the intersection of these two exemplary sides may be along, for example, a line extending normally to the optical axis of the shaped fiber optic tip (e.g., so that the side 301 is bifurcated horizontally into two planar sides having orientations differing by one degrees).

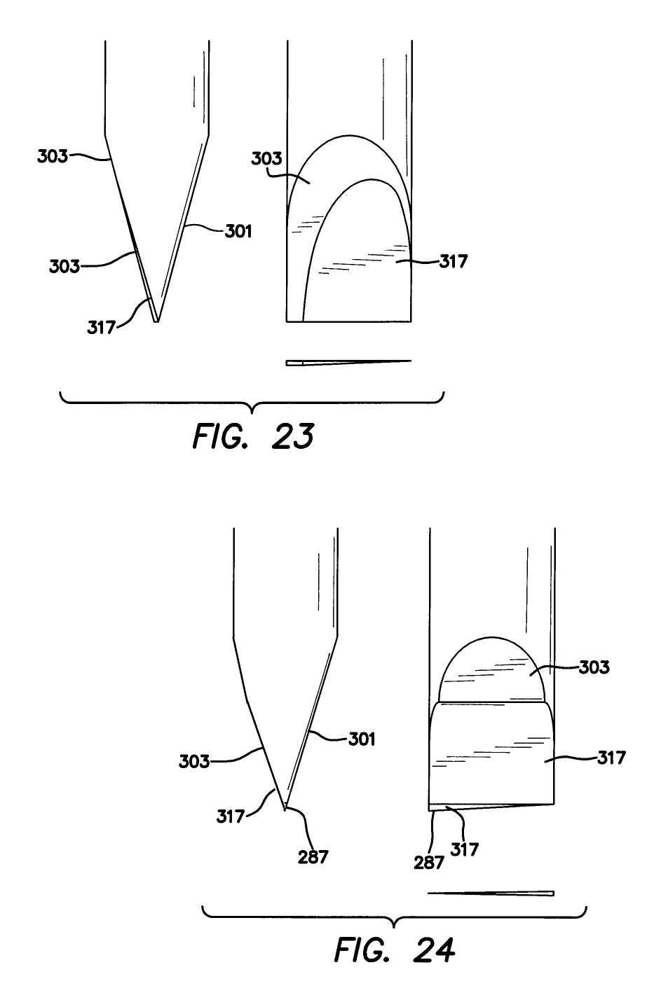

Each of the implementations of FIGS. 22-24 comprises one or more of a nonsymmetrical (e.g., not parallel or perpendicular to the optical axis or to another surface) surface a nonsymmetrical surface, and a nonsymmetrical edge. The construction of FIG. 22, which is intended to be to scale, comprises a distal edge 287 that is not perpendicular to the optical axis of the shaped fiber optic tip and further comprises either a distal edge having a relatively consistent thickness as shown in version A or a distal edge 287 having a varying thickness as shown in version B. The constructions of FIGS. 23 and 24, which are intended to be to scale, comprise sides 317 having surfaces that are not parallel the optical axis or to the surface 303 and further comprise distal edges 287 having varying thicknesses. A side 317′ is implemented in FIG. 24, as well.

On the subject of truncated distal surfaces, in FIG. 13 and others using reference designator character d or reference designator character w, the dimension of either of these variables can be, for example, from 100 to about 300 microns. In a typical example, the dimension of either reference designator character d or reference designator w can be about 300 microns. According to the present invention, a percentage of beams of laser radiation exit from the side-firing output end at relatively high angles (e.g., up to 90 degrees) with respect to the fiber optic axis.

For the truncated distal surface configurations such as depicted, for example, in FIGS. 13-15, according to an aspect of the present invention, beams of laser radiation exit from the side-firing output end at relatively high angles (e.g., up to 90 degrees) with respect to the fiber optic axis to form a ring 289 or opposing flanks 289′ in an irradiation pattern, and a percentage of beams of laser radiation exit along the optical axis of the fiber to form a centrally illuminated spot 291 or 294. Furthermore, in some embodiments, a dark or “blind spot” 293 or 296 may be formed in front of the side-firing output end such that the output beam pattern or illuminated area comprises a non-illuminated portion between the irradiated areas.

For the truncated distal surface configurations depicted, for example, in FIG. 14, according to an aspect of the present invention, more ((e.g., a majority, or in another embodiment about 85%, or in another embodiment about 95%, or in another embodiment substantially all) beams of laser radiation exit from one (e.g., a single one) side than another side single side of the side-firing output end at relatively high angles (e.g., up to 90 degrees) with respect to the fiber optic axis.

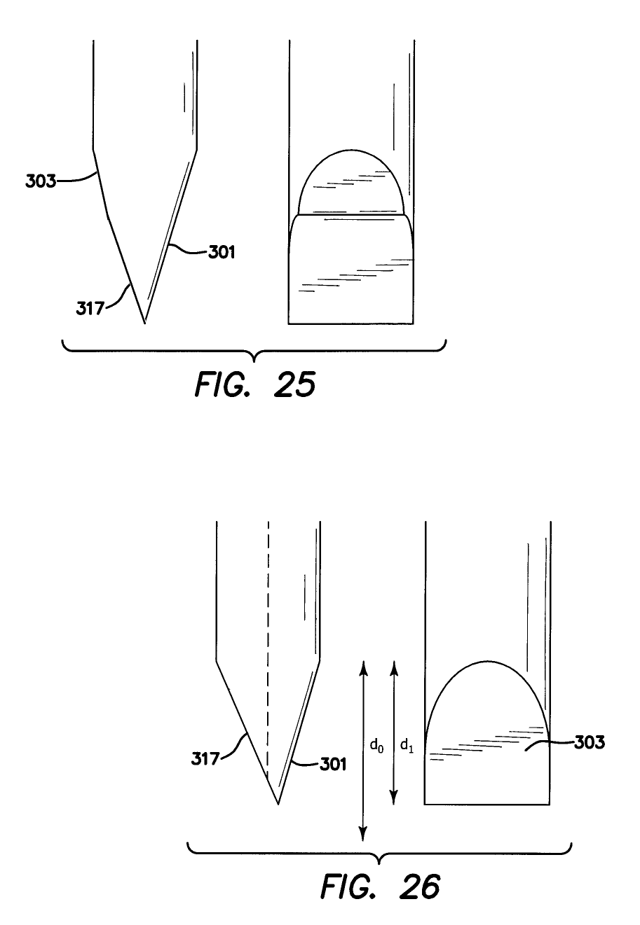

Regarding embodiments which do not implement truncated distal surface configurations, the members 301 and 303 may be generated, for example, by performing polishing of the sides to yield a structure having a relatively sharp (e.g., not flattened, or in another implementation not rounded) distal edge or blade which can result in an irradiation pattern without a (e.g., or with an attenuated, or with a dramatically attenuated) central or middle irradiated area (e.g., 291 or 294). The constructions of FIGS. 10, 11, 12, 12A, 12B, 16-27, 30, 32C, 33C and 33D correspond to a shaped fiber optic tip without a truncated distal surface, and, instead, with a distal end, ridge, edge (e.g., distal edge 287) or blade. The two sides 301 and 303 may be generated by polishing the respective areas all of the way down to form a distal edge 287. In another implementation, the two sides 301 and/or 303 may be full or partially formed by polishing, followed by polishing of the distal end, or of another area or areas, of the structure, followed by polishing of the two sides 301 and/or 303.

In FIG. 14, a side 317 is formed from an initial structure (e.g., that of FIG. 13) having a truncated distal surface. The distal end of the resulting structure, as shown in FIG. 4, still has a truncated distal surface, but a thickness t of the truncated distal surface of the resulting structure in FIG. 14 is less than a diameter d of the truncated distal surface of the beginning structure of FIG. 13. Although the thickness t is changed, a length d₁ of the resulting structure is preserved to be the same as the length d₁ of the initial structure depicted in FIG. 13.

The side 317 formed in connection with the structure of FIG. 15 from an initial structure (e.g., that of FIG. 13) having a truncated distal surface. The distal end of the resulting structure, as shown in FIG. 4, still has a truncated distal surface, but a thickness t of the truncated distal surface of the resulting structure in FIG. 14 is less than a diameter d of the truncated distal surface of the beginning structure of FIG. 13. Although the thickness t is changed, a length d₁ of the resulting structure is preserved to be the same as the length d₁ of the initial structure depicted in FIG. 13.

In FIG. 31, a structure is formed from, for example, an initial architecture such as depicted in FIG. 15. In one implementation, following formation of the FIG. 15 structure, a side is formed on side 303 for directing electromagnetic energy to side 301. The side can be formed to reside, for example, between the distal end (e.g., the truncated distal surface) of the side-firing output end and side 303, and, in the illustrated example, the thickness t from FIG. 15 can be wholly or partially preserved.

Regarding the side-firing output ends of the shaped fiber optic tips of FIGS. 10, 12A, 12B, and 13A-14D, any of these output ends may be modified or otherwise formed to have non-cylindrical, non-symmetrical, or otherwise irregular or different shapes, such as spherical, chiseled, or other light-intensity altering (e.g., dispersing) shapes and or surface(s), in additional embodiments. Also, regarding the side-firing output ends of the shaped fiber optic tips, any of these output ends further can be modified by removing more or less of the parts of the distally-disposed output ends to yield, for example, more or less truncated- or truncated-bevel distal ends that provide end-firing components.

Any of these tips and output ends may be modified or otherwise formed to have hollow interiors defining central fluid-delivery paths such as those described in connection with FIGS. 14a and 14b, and/or operated as such in whole or in part as described in connection with FIGS. 14a and 14b of the co-pending application referenced in the first paragraph of this disclosure. In exemplary implementations, the hollow interiors may be centered along fiber optic axes of the shaped fiber optic tips and/or may be aligned with what would otherwise be the planar output surfaces so that the planar output surfaces are not surfaces but rather are output openings of the hollow interiors.

In other implementations, the modified output ends (e.g., planar output surfaces) may have other orientations which are not perpendicular to the optical axes of the fiber optics, and in still further implementations the modified ends may comprise curved, rounded, or other non-planar surfaces, which may be wholly or partially frosted or otherwise etched.

The modified output ends (e.g., planar output surfaces) can generate output beam patterns similar to those described herein but with more or less filled center portions as a result of laser energy passing through, unrefracted, the planar output surfaces. The shapes and intensities of the filled center portions in the output beam patterns, resulting from implementations of the modified output ends, can be changed by changing characteristics (e.g., diameter and/or surface characteristics) as will be recognized by one skilled in the art in light of this disclosure.

The above-described embodiments have been provided by way of example, and the present invention is not limited to these examples. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. As iterated above, any feature or combination of features described and referenced herein are included within the scope of the present invention provided that the features included in any such combination are not mutually inconsistent as will be apparent from the context, this specification, and the knowledge of one of ordinary skill in the art. For example, any of the fiber optics, features thereof, or other features, including but not limited to the described side-firing output ends and the structures and methods referenced in U.S. application Ser. No. 11/033,441, may be used with any structure or process described or referenced herein, in whole or in part, in any combination or permutation. Accordingly, the present invention is not intended to be limited by the disclosed embodiments, but is to be defined by reference to the following additional disclosure in claims format.

1. A laser handpiece, comprising a shaped fiber optic tip having a proximal end, a distal end, an optical axis extending therebetween, and a side-firing output end having first and second members within the shaped fiber optic tip with each of the first and second members being oriented to direct distally traveling light within the shaped fiber optic tip toward a third member within the shaped fiber optic tip, so that the redirected light impinges on and is emitted from the third member in a direction distally and radially away from the optical axis, wherein a spatial distribution of electromagnetic radiation emitted from the side-firing output end has a relatively small component along the optical axis of the shaped fiber optic tip.

2. The laser handpiece as set forth in claim 1, each of the members comprising a surface of the side-firing output end.

3. The laser handpiece as set forth in claim 1, wherein the first member forms an angle of about 1 to about 10 degrees with the optical axis.

4. The laser handpiece as set forth in claim 1, wherein the second member forms an angle of about 20 to about 80 degrees with the optical axis and the third member forms an angle of about 1 to about 10 degrees with the optical axis.

5. The laser handpiece as set forth in claim 1, wherein the first member forms an angle of about 2.5 to about 3 degrees with the optical axis and the third member forms an angle of about 2.5 to about 3 degrees with the optical axis.

6. The laser handpiece as set forth in claim 1, wherein an angle formed by the first member with the optical axis is equal to an angle formed by the second member with the optical axis.

7. The laser handpiece as set forth in claim 1, wherein the distal end comprises a blade.

8. The laser handpiece as set forth in claim 1, wherein a thickness of the blade is about 100 to about 300 microns.

9. The laser handpiece as set forth in claim 1, the first member forming an angle with the optical axis that is less than an angle formed by the second member with the optical axis.

10. The laser handpiece as set forth in claim 1, the first member forming an angle of about 1 to about 10 degrees with the optical axis and the second member forming an angle of about 20 to about 80 degrees with the optical axis.

11. The laser handpiece as set forth in claim 1, the first member forming an angle of about 2.5 to about 3 degrees with the optical axis and the second member forming an angle of about 45 to about 60 degrees with the optical axis.

12. The laser handpiece as set forth in claim 1, each of the members comprising a planar surface.

13. The laser handpiece as set forth in claim 1, each of the members comprising a beveled surface of the side-firing output end

14. The laser handpiece as set forth in claim 1, further comprising a source of positive pressure coupled to the shaped fiber optic tip.

15. The laser handpiece as set forth in claim 14, the source of positive pressure being coupled to deliver fluid along a path, which is substantially parallel to the optical axis, to the shaped fiber optic tip.

16. The laser handpiece as set forth in claim 15, wherein the source of positive pressure and the path are configured to deliver the fluid to a vicinity of the shaped fiber optic tip as atomized fluid particles.

17. The laser handpiece as set forth in claim 16, wherein:
the source of positive pressure and the path are structured to place the atomized fluid particles into a volume in close proximity to the side-firing output end; and
the laser handpiece is constructed to deliver electromagnetic energy from an electromagnetic energy source into the atomized fluid particles in the volume to thereby expand the atomized fluid particles in such a way that when the volume is placed next to a target surface disruptive forces are imparted onto the target surface.

18. The laser handpiece as set forth in claim 17, wherein the fluid particles comprise water.

19. The laser handpiece as set forth in claim 18, wherein the target surface comprises tooth tissue.

20. The laser handpiece as set forth in claim 18, wherein the electromagnetic energy source comprises one of a wavelength within a range from about 2.69 to about 2.80 microns and a wavelength of about 2.94 microns.

21. A laser handpiece, comprising a shaped fiber optic tip having a proximal end, a distal end, an optical axis extending therebetween, and a side-firing output end comprising two surfaces within the shaped fiber optic tip wherein each of the two surfaces is oriented to direct distally traveling light within the shaped fiber optic tip toward another surface within the shaped fiber optic tip, so that the redirected light impinges on and is emitted from the other surface in a direction distally and radially away from the optical axis, wherein a spatial distribution of electromagnetic radiation emitted from the side-firing output end has a relatively small component along the optical axis of the shaped fiber optic tip.

22. The laser handpiece as set forth in claim 21, wherein the other surface forms an angle of about 1 to about 10 degrees with the optical axis.

23. The laser handpiece as set forth in claim 21, wherein one of the two surfaces forms an angle of about 1 to about 10 degrees with the optical axis.

24. The laser handpiece as set forth in claim 21, wherein the other surface forms an angle of about 2.5 to about 3 degrees with the optical axis and one of the two surfaces forms an angle of about 2.5 to about 3 degrees with the optical axis.

25. The laser handpiece as set forth in claim 21, wherein an angle formed by one of the two surfaces with the optical axis is equal to an angle formed by the other surface with the optical axis.

26. The laser handpiece as set forth in claim 21, wherein the distal end comprises a blade.

27. The laser handpiece as set forth in claim 21, wherein a thickness of the blade is about 100 to about 300 microns.

28. The laser handpiece as set forth in claim 21, a first surface of the two surfaces forming an angle with the optical axis that is less than an angle formed by the second surface of the two surfaces with the optical axis.

29. The laser handpiece as set forth in claim 21, the first surface forming an angle of about 1 to about 10 degrees with the optical axis and the second surface forming an angle of about 20 to about 80 degrees with the optical axis.

30. The laser handpiece as set forth in claim 29, the first surface forming an angle of about 2.5 to about 3 degrees with the optical axis and the second surface forming an angle of about 45 to about 60 degrees with the optical axis.

31. The laser handpiece as set forth in claim 21, the laser handpiece further comprising a source of positive pressure coupled to the shaped fiber optic tip.

32. The laser handpiece as set forth in claim 31, the source of positive pressure being coupled to deliver fluid along a path, which is substantially parallel to the optical axis, to the shaped fiber optic tip.

33. The laser handpiece as set forth in claim 32, wherein the source of positive pressure and the path are configured to deliver the fluid to a vicinity of the shaped fiber optic tip as atomized fluid particles.

34. The laser handpiece as set forth in claim 33, wherein the source of positive pressure and the path are structured to place the atomized fluid particles into a volume in close proximity to the side-firing output end; and
the laser handpiece is constructed to deliver electromagnetic energy from an electromagnetic energy source into the atomized fluid particles in the volume to thereby expand the atomized fluid particles in such a way that when the volume is placed next to a target surface disruptive forces are imparted onto the target surface.

35. The laser handpiece as set forth in claim 34, wherein the fluid particles comprise water.

36. The laser handpiece as set forth in claim 35, wherein the target surface comprises tooth tissue.

37. The laser handpiece as set forth in claim 35, wherein the electromagnetic energy source comprises one of a wavelength within a range from about 2.69 to about 2.80 microns and a wavelength of about 2.94 microns.
